(19) United States
(12) Patent Application Publication
(10) Pub. No.: US 2022/0323274 A1
(43) Pub. Date: Oct. 13, 2022

(54) A PERSONALIZED ACCESSORY FOR PROTECTION OF A BODY PART AND A MANUFACTURING METHOD OF THE SAID ACCESSORY

(71) Applicant: Fistula Protect, Gal Hostnik S.P., Gorica pri Slivnici (SI)

(72) Inventors: Gal HOSTNIK, Celje (SI); Anze VOLOVSEK, Preserje (SI)

(21) Appl. No.: 17/642,560

(22) PCT Filed: Sep. 16, 2020
(86) PCT No.: PCT/SI2020/050021

§ 371 Date: Mar. 11, 2022

(30) Foreign Application Priority Data
Sep. 20, 2019 (SI) P-201900177

(51) Int. Cl. A61F 15/00; B33Y 80/00; B33Y 10/00; B33Y 50/00; B29C 64/386; A61M 1/36

(52) U.S. Cl. CPC A61F 15/008; B33Y 80/00; B33Y 10/00; B33Y 50/00; B29C 64/386; A61M 1/3655; B29L 2031/753

(57) ABSTRACT

The present invention belongs to the field of medical accessories, more precisely to the field of accessories for protection against outer conditions such as impacts, high or low temperature and similar conditions that can harm a human being. The invention relates to a personalized accessory for protection of a body part, which comprises a protective element, which fits to the shape of the excrescence, a scar or a wound, and an adjustable part to which the protective part is attached, glued, sewed or inserted, wherein the adjustable part can be stretched or in any other way adjusted to the particular body part, which has to be protected. The said accessory is manufactured in the following way: first the body part with the injury is scanned or measured in order to obtain a 3D model of the scanned or measured body part. Then the protective element is made using a 3D printer or thermoforming with an adjustable mould, wherein the finished protective element is then attached to the adjustable part.

### FIELD OF THE INVENTION

The present invention belongs to the field of medical accessories, more precisely to the field of accessories for protection against outer influences such as impacts, high or low temperature and similar conditions that can harm a human being. The invention relates to a personalized accessory for protection of a body part, in particular to accessories for protection of AV fistulas that are usual in patients undergoing dialysis. The subject-matter of this invention is also a manufacturing method of the said accessory.

### BACKGROUND OF THE INVENTION AND THE TECHNICAL PROBLEM

Numerous people suffering from certain diseases are faced with scars, outgrowth (excrescence) and wounds on parts of a body that have to be protected in order to prevent infection, further injury or in order to improve the visual appearance of the affected body part. One of such outgrowths, which requires special attention, are arteriovenous (AV) fistulas in patients undergoing dialysis.

In order to ensure more efficient dialysis patients need an operatively altered blood vessel in one arm, so that the access to blood flow is easier. Operatively joined vein and artery is called arteriovenous (AV) fistula, its purpose is to enable a large volume of blood for efficient haemodialysis and for lowering chances for infection. Due to the AV fistula the patient is exposed to a significant risk, as in case of injury of the AV fistula the patient can quickly loose too much blood, which may be life-threatening.

Therefore, the technical problem, which is solved by the present invention, is a design of a personalized accessory for protection of AV fistulas and also all other similar outgrowths, scars or wounds, which are a consequence of a disease, injury or their treatments.

### STATE OF THE ART

Protective products after dialysis are mostly bandages around the arm, which are expensive, not environmentally friendly and potentially even dangerous, especially if the bandage is too tight or compresses the arm too strongly.

Companies that produce medical accessories and protective products offer only sleeves made of various materials, which are available in many sizes and colours, but are not adapted to the shape of patient's fistula, as they are mostly intended for hiding scars, preventing cuts or protecting against UV rays.

Document CN106963533 describes an adaptable fistula protection, which comprises a support device with an adjustable angle, and an inner sleeve. The support device has a base and a support plate, which is attached to the base with a hinge. The inner sleeve has a U-shaped groove and a zipper for opening and closing. This solution differs from the present invention, as it does not allow movement and does not allow use in everyday activities.

Patent application CN204072485 describes a sleeve for protecting fistulas, which comprises a front and a back sleeve, inside which a filling for arm protection and holes for air exchange are provided. The sleeve is via a rope placed around the shoulder and part of the back of a patient. This solution differs from the present invention as the shape of the sleeve filling is not adjusted to the shape of the fistula, but the shape of the sleeve is generic.

Document CN202960842 relates to a fixed device with a sleeve inserted in a fixed supporting frame, due to which the fistula is protected against outer pressures and injuries. This solution does not allow everyday use due to its rigid and non-transportable construction. Furthermore, the shape of the sleeve is generic.

### DESCRIPTION OF THE SOLUTION OF THE TECHNICAL PROBLEM

The essence of the accessory for protection of a body part according to the invention is in that the accessory has a protective part, which is adapted to the shape of the injury, preferably to the shape of an AV fistula, due to which it optimally protects the affected part. The personalized accessory for protection of a body part, such as knees, elbows, ankles, neck, fingers, etc. having an excrescence, a scar or a wound, and particularly AV fistula, thus comprises the protective element, which is adjusted to the shape of the excrescence, a scar or a wound, and an adjustable part to which the protective part is attached, glued, sewed or inserted, wherein the adjustable part can be stretched or in any other way adjusted to the particular body part, which has to be protected. The protective element may be made from any suitable material, particularly from plastics or rubber, while the adjustable part may be made from an elastic material such as nylon or spandex, any other textile, rubber, foam, 3D fibre mesh or liquid gel. It is important that the material of the adjustable part enables adjustment to the shape and the size of the body part to be protected. All materials have to comply with medical requirements and are preferably skin-friendly, i.e. without allergens. The protective element may be provided with holes for better breathability of the skin, wherein the holes may be made during the manufacturing process or later. Preferably both parts are coated with an antibacterial layer, which may be any suitable for such use, while at least one part of the accessory may be painted with a suitable paint to resemble skin. This improves the aesthetics of the protective accessory. The dimensions are arbitrary and depend on the body part, its shape and size as well as the size of the excrescence, scar or wound.

The personalized accessory according to the invention is manufactured in the following was: the body part with the injury is scanned or measured in order to obtain a 3D model of the scanned body part, preferably the width, length and height of the injury or the excrescence. The preferred method for obtaining the said measurements is scanning, which may be performed in any suitable way, preferably using an application, which works in a similar way as commercially available application “3D scanner pro”. The steps of the scanning and shaping the suitable file for 3D printing of the accessory are:
a) preparing the body part for scanning, wherein all clothes or elements are removed and a suitable measurement device such as a ruler or a tape measure is provided near the body part,
b) activation of the application, which scans at least a part of the selected body part, either by taking individual pictures (images), either by filming of the body part, wherein also manual taking of images or videos is possible independently from the application; the images or video may be taken with a suitable camera, photography camera, mobile phone, tablet or any other suitable equipment which can generate a suitable scan;
c) the scan obtained in step b) is preferably transformed into a suitable format file such as .obj or similar;
d) the obtained file in step c) is preferably processed, wherein name, number, slight adjustments and similar may be made in order to obtain a 3D model of the scanned body part;
e) based on the 3D model obtained in step d) the protective part for protection of the selected body part is generated with a suitable computer program, usually a .stl file is obtained, which can be transformed into a .gcode file needed for 3D printing.

When the file for the protective part in step e) is prepared, the file for 3D print or a file for any other suitable method, such as thermal forming, is sent to a suitable manufacturing device.

Then the protective element is made with a 3D printer, the said element corresponds to the previously obtained 3D model. Different methods of 3D printing may be used to manufacture the protective element, wherein the said methods are carried out according to usual protocols for selected materials or combination of suitable materials. Some of the possible methods are Poly Jetting, selective laser sintering (SLS), fused filament fabrication (FFF) or fused deposition modelling (FDM). Preferably 3D printer based on SLA and FDM/FFF processes is used, as these are easier to insert into the machine as well as cheaper and lighter for use. In the process of 3D printing the materials are provided in the shape of a thermoplastic filament, which is unwound during the process and lead through an injecting nozzle. The nozzle then melts the filaments and layers them onto the base plate or a support. The base plate and the nozzle is controlled with a computer, which helps in creation and adjustment of X, Y, Z coordinates during printing. The nozzle is above the base plate moved horizontally and vertically and thus creates a thin layer of plastics, which solidifies upon cooling and connects with the layer below it. The base plate is then lowered in order to enable formation of the next plastic layer. This process is continued until the whole plan is finished. Different materials belong to the suitable materials, such as thermoplastic polymeric materials, for example polycarbonate (PC), polyethylene (PE), polystyrene (PS), polyurethane (TPU), acrylonitrile butadiene styrene (ABS), polylactic acid (PLA), recycled plastic, reinforced polymeric materials with glass fibres, as well as rubber, resin, photopolymeric resin and metal materials such as aluminium, steel and titanium, which are applied during 3D printing in powder form.

For some of the mentioned materials, for example different kinds of rubber, plastics and polyurethane foam other methods for manufacturing may be used, such as thermoforming of the above mentioned materials using an adjustable physically adaptable mould. The mould is reset to the basic state before each new manufacturing process and is thereafter adjusted to the prepared 3D model of the injury. Thermoforming is a transformation of a polymeric material using heat to the selected shape based on the pre-prepared mould or the 3D model of the injury. The thermoforming process begins, when a computer or a suitable programme for recognition of files similar to files for 3D printing receives the modelled protection part.

The device for thermoforming comprises hydraulically or mechanically movable spikes or pistons arranged one next to the other at small distances (usually few millimetres or centimetres), so that they are able to reproduce the shape of the injury (mould), to which a layer of the material to be shaped will be provided. In the basic state the said spikes or pistons are in the same plane, while they will rearrange and vertically rise upon receiving the file, in order to form the required shape. Onto this shape achieved with spikes or pistons a layer of material is provided and above it:
the second part of the mould is moved closer, wherein this second part is fixed and serves as a heater to heat the polymeric material above the temperature needed for melting and reshaping of the material with regards to the mould,
or the second part of the mould is moved closer in order to serve as a negative which adjusts to the lower mould and compresses as well as heats the material provided in-between in order to reshape it.

The manufactured protective element is attached, glued, sewed or inserted onto the adjustable part, wherein the adjustable part may be stretched or in any other way adjusted to the size of the body part to be protected. The process of attachment to the adjustable part is the same regardless of the manufacturing method of the protective part. Some of the few possibilities are given below:

To the bottom part of the protective part, which is in contact with the skin, a mesh or a suitable breathable fabric is attached or glued, The said mesh or fabric may be coated with an antibacterial agent, while the whole protective part may be attached, glued or sewn onto the adjustable part, which is equipped with attachment tapes with loops and hooks in order to attach the accessory to the body part. Instead of the loops and hooks tape any other known solution for attaching accessories to a body part known in medical equipment may be used.

The protective part may be entirely covered with the adjustable part or the latter is attached to the edges of the protective element, so that the protective element is visible. In case the protective part is attached to the adjustable part with tapes with loops and hooks, additional stabilization may be provided by sewing.

In case of thermoforming of the protective part, the later may be sewn at the sides onto the pre-prepared adjustable part, wherein the protective part does not need the aforementioned mesh or fabric.

Until now, known protective accessories have not been personalized, especially not with the use of scanning technologies and 3D printing. These techniques enable optimal adjustment to the shape of a fistula and thus reliable protection against injuries. The personalized accessory for protection of a body part according to the invention enables:
protection against cuts and impacts,
elasticity and comfort,
compression for stopping bleeding in case of AV fistulas,
protection against UV radiation, and
covering wounds, scars, or excrescences due to aesthetic reasons.

The accessory may be easily placed and fastened to the affected body part and can be used in everyday activities, sports and other actions. Use of the accessory according to the invention in dialysis patients enables an optimal pressure to the fistula following the dialysis, wherein it also functions as a compression for stopping the bleeding after dialysis. Most importantly it ensures safety during work, physical or recreational activities.

Other embodiments clear to the person skilled in the art are possible in the range of invention as described herein and defined in the claims. The discussed exemplary embodiments thus do not limit the essence of the invention.

The personalized accessory for protection of a body part and a manufacturing method thereof will be described in further detail based on exemplary embodiments and figures, which show:

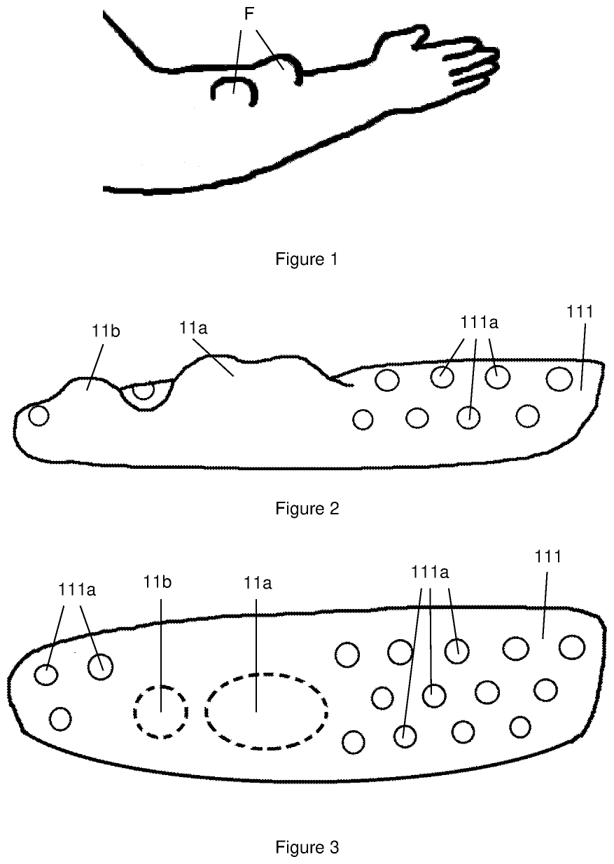

FIG. 1 Sketch of the AV fistula on a patient's arm

FIG. 2 protective part of the accessory according to a possible embodiment

FIG. 3 Plan view of the protective part of the accessory according to a possible embodiment

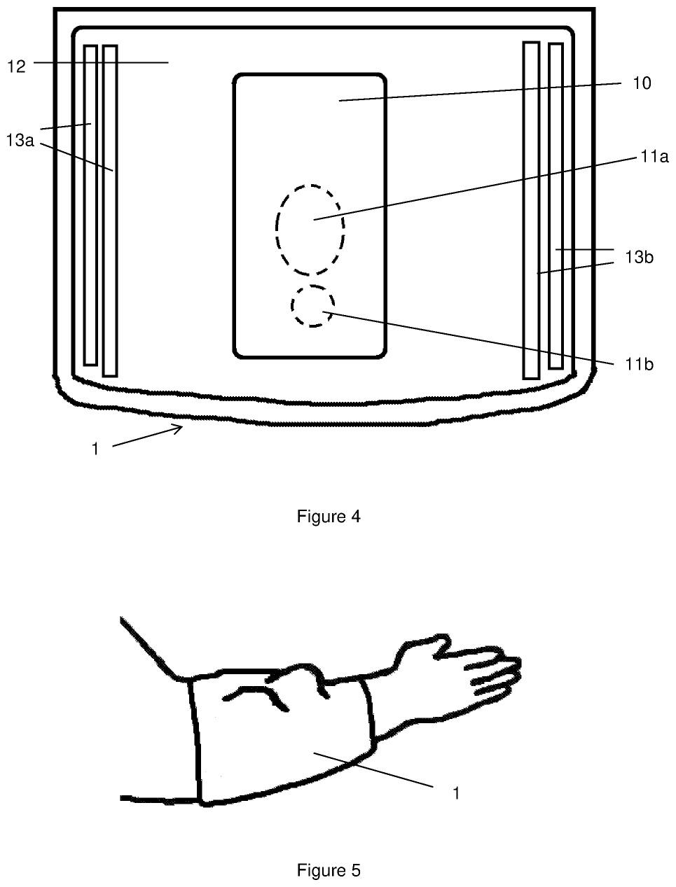

FIG. 4 Integrated protective part into the adjustable part according to a possible embodiment

FIG. 5 Sketch of the accessory for protection of an AV fistula on an arm

FIG. 1 shows a sketch of a patient's arm with an AV fistula (excrescences F), which has to be protected. According to the preferred embodiment the arm is scanned with a mobile application, which is performed according to the following steps:
a) preparing the body part for scanning, wherein all clothes or elements are removed and a suitable measurement device such as a ruler or a tape measure is provided near the body part,
b) activation of the application, which scans at least a part of the selected body part, either by taking individual pictures (images), either by filming of the body part, wherein also manual taking of images or videos is possible independently from the application; the images or video may be taken with a suitable camera, photography camera, mobile phone, tablet or any other suitable equipment which can generate a suitable scan;
c) the scan obtained in step b) is preferably transformed into a suitable format file such as .obj or similar;
d) the obtained file in step c) is preferably processed, wherein name, number, slight adjustments and similar may be made in order to obtain a 3D model of the scanned body part;
e) based on the 3D model obtained in step d) the protective part for protection of the selected body part is generated with a suitable computer program, usually a .stl file is obtained, which can be transformed into a .gcode file needed for 3D printing

There are several excrescences, therefore each of them and their characteristics are precisely described and defined prior to manufacturing, so that the accessory and especially its protective part corresponds and fits on the injury. This is the reason for preparation of the 3D model of the AV fistula as described above.

FIGS. 2 and 3 show the protective part 11 of the accessory 1, wherein the raised parts 11a in 11b fit to the excrescence F of the fistula. The base part 111 of the protective element 11 is provided with holes 111a that ensure air flow and skin breathing. The protective part as shown may be made according to any of the above described methods; preferably it is made using 3D printing based on the model of the scanned patient's arm. The protective element may be made from any suitable material, especially from plastic or rubber; it may be covered with a mesh or any other breathable material, while it can also be additionally provided with a layer of an antibacterial agent.

FIG. 4 shows the accessory 1 according to a possible embodiment, the accessory comprising;
the protective element 11, which fits (corresponds) to the shape of the excrescence, scar or wound, wherein the protective element 11 is sewn into the middle part 10 of the accessory, and
the adjustable part 12, onto which the protective part 11 is attached, preferably sewn,
   wherein the adjustable part may be stretched or in any other way adjusted to the shape of the body part with the AV fistula. The adjustable part 12 is equipped with attachment tapes 13a, 13b that enable attachment to the patient's body part, in this embodiment to the patient's arm. The adjustable part 12 is preferably made from an elastic fabric such as nylon and spandex, while the dimensions are arbitrary. It may cover the part form the wrist to the shoulder or a smaller part, for example only to the elbow. The finished accessory 1 for protection of the AV fistula is placed on the arm of a patient as shown in FIG. 5, so that it covers the part of the arm from the wrist to the elbow.

1. A personalized accessory for protection of a body part comprising:
a protective element, which fits to the shape of the excrescence, a scar or a wound on a body part, wherein the protective element is made based on measurements and/or scanning the injury, excrescence, scar or wound; and
an adjustable part, to which the protective element is attached, glued, sewn or inserted, wherein the adjustable part may be stretched or in any other way adjusted to the size of the body part to be protected.

2. The personalized accessory for protection of a body part according to claim 1, characterized in that the protective element is made from any suitable material, especially from plastics or rubber, while the adjustable part is made from an elastic fabric such as nylon and spandex or rubber, foam or 3D fibre mesh.

3. The personalized accessory for protection of a body part according to claim 1 or claim 2, characterized in that the protective part is made from a material selected in the group consisting of thermoplastic polymeric materials, for example polycarbonate (PC), polyethylene (PE), polystyrene (PS), polyurethane (TPU), acrylonitrile butadiene styrene (ABS), polylactic acid (PLA), recycled plastic, reinforced polymeric materials with glass fibres, as well as rubber, resin, photopolymeric resin and metal materials such as aluminium, steel and titanium.

4. The personalized accessory for protection of a body part according to any of the preceding claims, characterized in that the protective element may be equipped with holes for improved skin breathability, said holes may be made during manufacturing or later.

5. The personalized accessory for protection of a body part according to any of the preceding claims, characterized in that a bottom part of the protective part, which is in contact with skin, is provided with a mesh or a suitable breathable fabric.

6. The personalized accessory for protection of a body part according to any of the preceding claims, characterized in that both parts have an antibacterial coating.

7. A manufacturing method of the personalized accessory according to any of the preceding claims, the method comprising the following steps:
a) scanning or measuring the body part with the injury, based on which a 3D model of the scanned body part is obtained, preferably width, length and height of the injury or the excrescence,
b) preparation of the 3D model of the measured or scanned body part obtained in the previous step,
c) making of the 3D model of the protective element, which fits to the 3D model obtained in step b),
d) manufacturing of the protective element using any suitable method, preferably with 3D printing or thermoforming, and
e) manufacturing of the accessory with the protective part made in step d).

8. The manufacturing method according to the preceding claim, characterized in that the steps of scanning and shaping a suitable file for manufacturing the accessory are as follows:
i. preparing the body part for scanning, wherein all clothes or elements are removed and a suitable measurement device such as a ruler or a tape measure is provided near the body part,
ii. activation of the application, which scans at least a part of the selected body part, either by taking individual pictures (images), either by filming of the body part, wherein also manual taking of images or videos is possible independently from the application; the images or video may be taken with a suitable camera, photography camera, mobile phone, tablet or any other suitable equipment which can generate a suitable scan;
iii. the scan obtained in step ii) is preferably transformed into a suitable format file such as .obj or similar;
iv. the obtained file in step iii) is preferably processed, wherein name, number, slight adjustments and similar may be made in order to obtain a 3D model of the scanned body part;
v. based on the 3D model obtained in step iv) the protective part for protection of the selected body part is generated with a suitable computer program, usually a .stl file is obtained, which can be transformed into a .gcode file needed for 3D printing.

9. The manufacturing method of the personalized accessory according to any of the claims from 7 to 8, characterized in that the file for the protective part in step v) is prepared and then the file for 3D printing or the file for any other suitable method such as thermoforming is sent to a suitable manufacturing device.

10. The manufacturing method of the personalized accessory according to any of the claims from 7 to 9, characterized in that for manufacturing of the protective element different methods of 3D printing may be used, such as Poly Jetting, selective laser sintering (SLS), fused filament fabrication (FFF) or fused deposition modelling (FDM), preferably 3D printing based on SLA and FDM/FFF processes.

11. The manufacturing method of the personalized accessory according to any of the claims from 7 to 9, characterized in that for manufacturing of the protective element thermoforming using a physically adjustable mould is used, wherein the said mould is returned to its basic state before each new manufacturing, the basic state then being adjusted with regards to the prepared 3D model of the injury.

12. The manufacturing method of the personalized accessory according to claim 11, characterized in that the device for thermoforming comprises hydraulically or mechanically movable spikes or pistons arranged one next to the other at small distances, usually few millimetres or centimetres, so that they can be able to reproduce the shape of the injury, to which a layer of the material to be shaped will be provided; wherein in the basic state the said spikes or pistons are in the same plane, while they are arranged to rearrange and vertically rise upon receiving the file in order to form the required shape and wherein a layer of material is provided over the shape formed by the spikes or pistons; and above it:
the second part of the mould is moved closer, wherein this second part is fixed and serves as a heater to heat the polymeric material above the temperature needed for melting and reshaping of the material with regards to the mould,
or the second part of the mould is moved closer in order to serve as a negative which adjusts to the lower mould and compresses as well as heats the material provided in-between in order to reshape it.

13. The manufacturing method of the personalized accessory according to any of the claims from 7 to 12, characterized in that the manufactured protective element is attached, glued, sewed or inserted on the adjustable part.

14. The manufacturing method of the personalized accessory according to any of the claims from 7 to 13, characterized in that the protective element may be entirely covered with the adjustable part or the latter is attached to the edges of the protective element, which is thus visible.
